(19) United States
(12) Patent Application Publication
(10) Pub. No.: US 2026/0358205 A1
(43) Pub. Date: Nov. 12, 2026

(54) A SAMPLING TOOL FOR SAMPLING REBURNED LIME FROM A LIME KILN, AND AN APPARATUS AND A METHOD FOR DETERMINING AMOUNT OF RESIDUAL CARBONATE IN REBURNED LIME SAMPLED FROM A LIME KILN

(71) Applicant: ANDRITZ OY, Helsinki (FI)

(72) Inventors: Ossi AALTONEN, Lahti (FI); Airi ANTIKAINEN, Helsinki (FI); Mika MUSSALO, Kotka (FI); Antti OKSMAN, Helsinki (FI); Jani RÄSÄNEN, Mikkeli (FI); Tuomo VEHVILÄINEN, Varkaus (FI)

(21) Appl. No.: 19/135,037

(22) PCT Filed: Nov. 17, 2023
(86) PCT No.: PCT/FI2023/050636

§ 371 Date: Jun. 2, 2025

(30) Foreign Application Priority Data
Dec. 2, 2022 (FI) 20226072

(51) Int. Cl. G01N 1/20; G01N 1/08; G01N 1/28; G01N 33/38

(52) U.S. Cl. CPC G01N 1/20; G01N 1/08; G01N 1/286; G01N 33/38; G01N 2001/2866

(57) ABSTRACT

A sampling tool (10) for sampling reburned lime from a lime kiln, the sampling tool includes a holding device (11) for holding the sampling tool (10) in the lime kiln during the sampling, and a preparation compartment (12) having
a sample inlet (13) for receiving a sample from the lime kiln into the preparation compartment (12),
a sample outlet (14) for unloading the sample out from the preparation compartment (12), and
a sieving arrangement (15) for limiting the sizes of the particle in the sample to a particle size range.

### FIELD OF THE INVENTION

The present invention relates to a sampling tool for sampling reburned lime from a lime kiln. Furthermore, the present invention relates to an apparatus and a method for determining amount of residual carbonate in reburned lime sampled from a lime kiln.

### BACKGROUND OF THE INVENTION

In a lime kiln lime mud (CaCO₃) is regenerated into reburned lime (CaO) with heat. Quality key performance indicator for burned lime in this process is the proportion of residual carbonate (CaCO₃%) within the reburned lime.

Traditionally proportion of the residual carbonate is measured by taking samples manually from the burned lime at the lime kiln and by analyzing the samples in laboratory by using acid to determine amount of residual carbonate within the reburned lime.

A drawback of this known method is that it takes operators time during the production since they must carry out the sampling among their other tasks. It is also hazardous for the operators since reburned lime taken from the lime kiln is very hot (up to 1000 °C) as well as corrosive and irritating.

### SUMMARY OF THE INVENTION

The object of the present invention is to provide a sampling tool for sampling reburned lime sample from a lime kiln, and an apparatus and a method by means of which lime sampling, sample handling and analysis of the lime samples can be carried out quicker without manual work and by means of which exposure to hazards is reduced.

The above-described problem is solved by the present invention because the sampling tool according to the present invention has a preparation compartment which can be used for sampling the reburned lime sample from a lime kiln and screening of the sample when the sampling tool is within the lime kiln as well as for unloading the sample to further processing (e.g., to the grinding device for grinding the sample), and because these sample preparation phases can be carried out by using e.g., a remote controlled or automated sampling tool moving device remotely, thus avoiding the manual sampling and sample preparation work requiring operators working time and causing stress and safety risks. More specifically, the sampling tool according to the invention is characterized by what have been described in the independent claim 1. An apparatus for determining amount of residual carbonate in reburned lime sampled from a lime kiln is characterized by what have been described in the independent claim 14. A method for determining amount of residual carbonate in reburned lime sampled from a lime kiln is characterized by what have been described in the independent claim 17. The dependent claims 2 to 13 present some advantageous embodiments of the sampling tool according to the invention, the dependent claims 15 and 16 of the apparatus according to the invention and the dependent claim 18 of the method according to the invention.

An advantage of the present invention is that the sampling tool does not require tool change because all phases of the sampling can be carried out by using the same sampling tool, which accelerates the testing process and creates cost savings. Another advantage is that the sampling tool according to the present invention enables automatization of the testing by applying e.g., an industrial robot such that it provides high level of flexibility with respect to layout (compared to e.g., large conveyor systems). Further advantage is that the sampling tool of the present invention allows to do all the required sample handling tasks by using a single device which minimizes the required amount of separate equipment. This makes testing, among others, more straightforward and cost effective. Automatization and straightforwardness make sampling process quicker which allows application of higher analyzing frequency. Higher analyzing frequency enhances data resolution and enables the operators to control the lime reburning process more precisely. In addition, when applying the invention, due to automatization the operators are not required to carry out any of the sampling tasks manually, which relieve them to focus their primary work, which, among others, lowers the stress experienced due to the work. Also, because the robotized sampling is possible by using the sampling tool according to the invention, at least most of the above-described safety hazards relating to manual sampling can be eliminated.

### BRIEF DESCRIPTION OF THE DRAWINGS

In the following, the invention will be described in more detail with reference to the appended drawings in which:

FIG. 1 shows an embodiment of a sampling tool according to the invention seen obliquely from above,

FIG. 2 shows a side view of the sampling tool shown in the FIG. 1 in a position wherein its analysis compartment is in upright position,

FIG. 3 shows a rear view of the sampling tool shown in the FIGS. 1 and 2, and

FIG. 4 shows a front view of the sampling tool shown in the FIGS. 1 to 3.

### DETAILED DESCRIPTION OF SOME ADVANTAGEOUS EMBODIMENTS

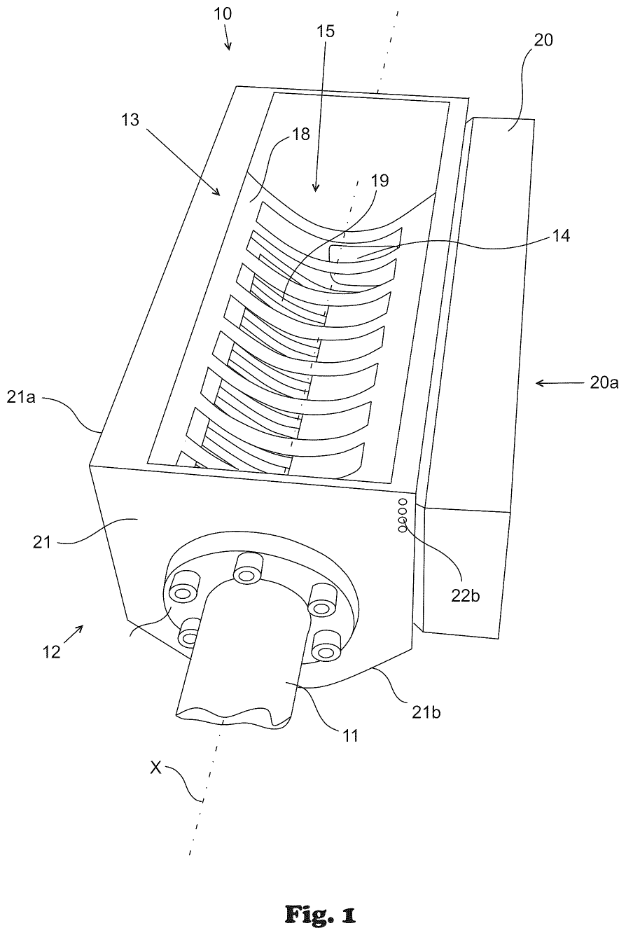

An embodiment of a sampling tool 10 shown in the FIGS. 1-4 is suitable for sampling lime samples from several different positions of a lime kiln. Most typically the lime samples are taken from a chute of a lime kiln but sampling from other positions e.g., from channels and conveyors of the lime kiln, or from inside the lime kiln, is also possible. Therefore, when it is used hereinbelow generic definition that the sample is taken or sampled from a lime kiln it is meant that sample may be taken from any of these places.

The embodiment of the sampling tool 10 shown in the FIGS. 1-4 comprises holding device 11 for holding the sampling tool 10 in a lime kiln during the sampling. Holding device 11 for holding the sampling tool 10 may be e.g., elongated arm-like element which is preferably so long that it extends from outside the lime kiln to the position inside the lime kiln wherein the sampling takes place. The embodiment of FIGS. 1-4 comprises further a preparation compartment 12 having sample inlet 13 for receiving a sample from the lime kiln, a sample outlet 14 for unloading the sample out from the preparation compartment 12 as well as a sieving arrangement 15 to limit the particle size range of the sample. In case of the embodiment of FIGS. 1-4 the sieving arrangement 15 is capable to screen from the sample particles having size over a predetermined upper particle size limit Pmax and a predetermined lower particle size limit Pmin. However, in some embodiments the sieving arrangement may be such that it is capable to screen from the sample only the particles having size over the predetermined upper particle size limit Pmax (i.e., such that sample accepted by the sieving arrangement contains sample particles having particle size between 0 to Pₘₐₓ), or such that it is capable to screen from the sample only the particles having size under the predetermined lower particle size limit Pₘᵢₙ (i.e., such that sample accepted by the sieving arrangement contains sample particles having particle size between Pₘᵢₙ to size which still fits into the preparation compartment through the sample inlet).

In the embodiment shown in the FIGS. 1-4 and wherein the sieving arrangement 15 is capable to screen from the sample particles having size over a predetermined upper particle size limit Pmax and under the predetermined lower particle size limit Pₘᵢₙ the particle size is determined by the smallest diameter of the sieve opening of a first sieve 18 or a second sieve 19 of the sieving arrangement 15. More specifically, the diameter of the sieve opening defines the dimension of the sample particle which fits through the first sieve 18 or the second sieve 19. If the sieve opening has constant dimensions in all directions, it defines the largest sample particle that can pass the sieve. However, if the sieve openings dimensions vary (e.g., the sieve opening is a groove-like) then it defines the largest minimum dimension of the sample particle that may pass the sieve as the case is with the first sieve 18 and the second sieve 19 of the embodiment of FIGS. 1-4. In case of the sampling tool shown in the FIGS. 1-4 preferred value for Pₘₐₓ is 25 mm and for Pₘᵢₙ 5 mm. However, these values can vary in different embodiments of the invention. Furthermore, in such embodiments wherein the sieving arrangement is capable to screen from the sample particles having size over a predetermined upper particle size limit Pₘₐₓ, or under the predetermined lower particle size limit Pₘᵢₙ, there may be only one sieve arranged to screen from the sample the particles having the respective particle size.

In the embodiment of FIGS. 1-4 the sample inlet 13 is an opening at the top of the preparation compartment 12, preferably being enough large and having shape that when the sampling tool 10 is placed, for instance under the chute of the lime kiln, lime can be received into the preparation compartment 12. In case of embodiment shown in the FIGS. 1-4, sample inlet 13 is formed by open top side of the preparation compartment 12. In the other embodiments it may alternatively include only part of the top side. Furthermore, in some other embodiments the sample inlet may include additional element or elements and/or can be such that it allows taking the sample, e.g., from any suitable position inside the lime kiln instead of under the chute.

The sampling tool 10 shown in the FIGS. 1 to 4 comprises also a cooling arrangement 16 arranged within the preparation compartment 12 to speed up the cooling of the sample in the preparation compartment 12. The lime sample taken from the lime kiln is very hot, typically it has temperature around 1000 °C. Thus, it is important to cool the sample down after the sample has been screened in the lime kiln as quickly as possible to enable forthcoming phases, i.e., grinding and analysis of the sample without wasting time.

The first sieve 18 and the second sieve 19 have been arranged into the preparation compartment 12 such that the first sieve 18 rejects sample particles having size over a predetermined upper particle size limit Pₘₐₓ and a second sieve 19 sample particles having size under a predetermined lower particle size limit Pₘᵢₙ. The sample inlet 13 is above the first sieve 18 and the sample space 17 is a space between the first sieve 18 and the second sieve 19. Thus, when the screening is carried out the sample particles having size between the predetermined upper particle size limit Pₘₐₓ and lower particle size limit Pₘᵢₙ remains into the sample space 17.

The sample outlet 14 is in the embodiment shown in the FIGS. 1 to 4 an opening arranged to the preparation compartment 12 at the position of the sample space 17. More specifically, it is formed into the side wall 21 at the front end of the preparation compartment 12 as it can be seen from FIG. 4. Thus, the screened sample can be unloaded by tilting the sampling tool 10 in a slanted angle such a way that sample outlet 14 is directed downwards. In such embodiment which has only one sieve for screening the particles having size over a predetermined upper particle size limit Pmax or under the lower particle size limit Pmin there may be only one sieve in the preparation compartment. In such case the only one sieve is located between the sample inlet and an impermeable bottom wall of the preparation compartment through which the sample particles accepted by the only one sieve cannot escape. Thus, in such embodiment the sample space is in within the sidewall between the only one sieve and the bottom wall. The sample outlet may be located similarly as it is in embodiment shown in the FIGS. 1-4.

To accommodate the sample during its analysis the sampling tool 10 may comprise an analysis compartment 20 being capable of receiving the sample from outside of the sampling tool 10. In the embodiment shown in FIGS. 1-4 the analysis compartment 20 is a tray attached outside of preparation compartment 12 to the side wall 21 of the preparation compartment 12. Thus, it has only bottom wall and side wall and it is open from its top end to receive the sample to be analyzed. However, in some other embodiments the analysis compartment may not be part of the sampling tool but can be separate from it. Thus, it may be arranged e.g., in connection with a separate analysis device which is exchanged to the sampling tool moving device so that the separate analysis device replaces the part of the sampling tool containing the preparation compartment, and into which the prepared sample is loaded before analysis. Loading is most typically realized so that sample is received from the grinding device wherein the screened and cooled sample is unloaded from the preparation compartment after screening. Thus, in an embodiment of sampling tool which has not its own analysis compartment the analysis compartment is not present during the preparation phase, and in the other hand, the preparation compartment is not present during the analysis phase. Nevertheless, there may be also such embodiment where the analysis compartment is detachably attached to the sampling tool and thus in case of such embodiment the analysis of the sample can be carried out by using a sampling tool with the analysis compartment or by using separate device comprising an analysis compartment.

As can be seen from FIGS. 1, 3 and 4, in the embodiment of FIGS. 1-4 the analysis compartment 20 has been attached to the side wall 21 of the preparation compartment 12 in such orientation that its open end 20a opens perpendicularly to the direction in which the sample is received to the preparation compartment 12 through the sample inlet 13 (i.e., the sample to the analysis compartment is received perpendicularly to the receiving direction of the sample into the preparation compartment 12). This is advantageous since due to this feature previously analyzed sample is unloaded from the analysis compartment 20 at the same moment when the sampling tool 10 is turned in the position wherein the preparation compartment 12 is capable for receiving next sample to be prepared through the sample inlet 13. In other embodiments wherein the sampling tool includes an analysis compartment the angle between the open end of the analysis compartment and the said receiving direction of the sample into the preparation compartment may vary being some other than a perpendicular angle. Thus, it can be, for instance any angle, preferably between 30° to 120°, so that analyzed, previous sample is correspondingly unloaded when the sampling tool is turned in to the position in which preparation compartment 12 is ready for receiving the next sample to be prepared.

The preparation compartment 12 of the sampling tool 10 shown in the FIGS. 1-4 comprises a side wall 21. Both, the first sieve 18 and the second sieve 19 comprise sieve plates 18a and 19a attached within the side wall at a distance from each other. The side wall 21 has upper edge 21a and lower edge 21b. As described above, in the embodiment of FIG. 1-4 there is no top wall in the preparation compartment 12 whereas it is open from its top side. Thus, the top side of the preparation compartment 12 forms the sample inlet 13 for receiving the sample. The sieve plate 18a of the first sieve 18 is in between the upper edge 21a and lower edge 21b of the side wall 21 hence forming to the preparation compartment an “intermediate floor”. The sieve plate 18a supports the sample when it is received into the preparation compartment 12 from the lime kiln through the sample inlet 13. The sieve plate 19a of the second sieve 19 is at the lower edge 21b of the side wall 21. However, in other embodiments like this, it may be also placed to some position between the sieve plate of the first sieve and the lower edge of the side wall such that appropriate space for sample particles passed through the first sieve is left between the sieve plate of the first sieve and sieve plate of the second sieve. The sieve plate 19a of the second sieve 19 forms in the sampling tool 10 the sole bottom wall to the preparation compartment 12. Thus, the particles of the sample having particle size below the predetermined lower particle size limit Pₘᵢₙ drop out from the preparation compartment 12 through the sieve plate 19b of the second sieve 19. In such embodiment wherein the mere sieve is a sieve for screening the particles having particle size under lower particle size limit Pₘᵢₙ the sieve may have only one sieve plate forming the bottom wall of the preparation compartment. In the embodiment of the FIGS. 1-4 the cooling arrangement 16 comprises a cooling medium channel 22 arranged within the preparation compartment 12. The cooling medium channel 22 is formed, in this embodiment, in the side wall of the preparation compartment by forming the side wall 21 double cover layers in between of which there is a hollow space as can be seen from FIG. 4. The hollow space is divided in sections which have been divided from each other by intermediate walls 22a that have been arranged within the side wall 21 such that cooling medium flows in all sections of the cooling medium channel 22. Thus, the cooling medium chills the whole side wall 21 when it flows through the cooling medium channel 22. Since, in this embodiment, the cooling medium is air, it is not necessary to return it back to its source whereas it can be released to atmosphere. This happens through the cooling medium outlets 22b shown in the FIGS. 1 and 4. In the other embodiments the cooling medium can be some other fluid that is suitable for cooling the sampling tool after sampling. However, air is advantageous because any kind of equipment for storage and return it back to any storage is not required as the case is with the other cooling mediums. This makes cooling arrangement simpler and cheaper. Furthermore, in such system it is possible to utilize existing compressed air supply system such as the lime kiln's compressed air supply system.

In the embodiment of FIGS. 1-4 the holding device 11 is an elongated arm-like element which has a first end 23 and a second end 24. In the second end there is a flange 24a by means of which the holding device can be attached to, or can be hold by a tool moving device, such as by an arm of an articulated robot. At the first end 23 there is a flange 23a by means of which the holding device 11 is secured to the front end of the preparation compartment 12. The holding device 11 is preferably designed so, that sampling tool 10 extends through a gate or hatch into the lime kiln such that tool moving device remains advantageously as much as possible outside the lime kiln. In this embodiment, the holding device comprises further a connecting conduit 25 connected to the cooling medium channel 22 of the preparation compartment 12 and a connector for connecting the connecting conduit 25 to a cooling medium supply device. Since the cooling medium is, in this case, air the connector can be e.g., pressure air connector. In the other embodiments cooling medium can be e.g., some liquid or other gas.

At least, in case when some liquid is used as a cooling medium, the system includes all the necessary equipment such as a storage container (e.g., a reservoir) for cooling liquid from which the cooling liquid is pumped to the sampling tool and into which the liquid is returned from the sampling tool after it has been circulated in the cooling channel of the sampling tool. In such case there could be also e.g., a heat exchanger or cooler for cooling the liquid before it is returned to the storage container.

When preparing the sampling tool according to FIGS. 1 to 4 to the lime sampling the sampling tool 10 is hold from its holding device 11 or the holding device 11 is attached to a tool moving device from its flange 24a at its second end 24 fixedly. The tool moving device may be e.g., some suitable manipulator by means of which the sampling tool 10 can be moved into the lime kiln for taking a sample, and from the lime kiln to the other devices needed when carrying out the analysis. Preferably, the tool moving device is an articulated robot (having, e.g., six degrees of freedom) since by it the sampling tool can be moved most freely in desired positions and locations during the sampling phase and analysis phase. Articulated robot is also advantageous because it can be programmed for producing suitable screening movements to accomplish the screening e.g., in a lime drop chute inside the lime kiln. Also, the connector of the cooling arrangement and the cooling medium supply device may be prepared so that the sampling tool can be connected to the cooling medium supply device immediately after the sampling tool 10 is taken out from the lime kiln. Since the cooling medium used in embodiment of FIGS. 1 to 4 is air, the cooling medium supply device is some suitable air supply device such as e.g., a compressed air supply system of the plant.

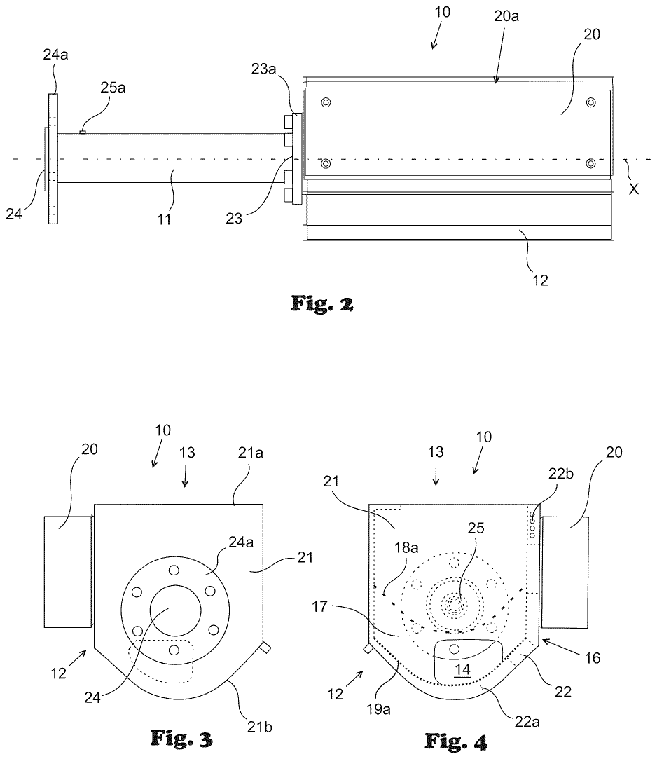

Sampling and determination of the amount of residual carbonate in a reburned lime sample by the sampling tool 10 according to FIGS. 1 to 4 can be divided e.g., in following phases:
1. The sampling tool 10 is inserted in lime drop chute, or in other position in the lime kiln and sample is collected into the preparing compartment 12 of the sampling tool 10,
2. Particles of the lime over the predetermined upper size limit Pₘₐₓ are screened out by the first sieve 18. During screening, the sampling tool 10 is inside the chute and screening movement carried out by twisting the arm of the robot (or by respective part of the other tool moving device) back and forth causing the sampling tool 10 to turn correspondingly in respect of its longitudinal axis X. This results that reject (i.e., sample particles having size above the predetermined upper particle size limit Pₘₐₓ) drops out from preparation compartment 12. Accept, in this phase (i.e., sample particles having size under the predetermined upper particle size limit Pₘₐₓ) goes through sieve plate 18a of the first sieve 18 to the sample space 17.
3. Particles under the predetermined lower particle size limit Pₘᵢₙ are screened out by the sieve plate 19a of the second sieve 19. This happens to the sample particles that have passed the sieve plate 18a of the first sieve 18, i.e., which are in the sample space. Reject (i.e., sample particles having size under the predetermined lower particle size limit Pₘᵢₙ) formed in this phase of the screening goes through sieve plate 19a of the second sieve 19 and drops back into the lime kiln. Accept, (i.e., the sample particles having size between the upper particle size limit Pₘₐₓ and the lower particle size limit Pmin) remain inside the sample space 17.
4. The sampling tool 10 is moved to the cooling station were cooling air supply is connected to the holding device 11 which conducts it to the cooling channel 22 of the preparation compartment 12. Thus, the preparation compartment's 12 side wall 21 is cooled, and hence also the sample inside the sample space 17 cools down. As the cooling is carried out with the cooling arrangement 16 through the side wall 21 of the preparation compartment 12 and not e.g., by direct blowing, as the case is when applying presently known methods, the sample is not dusted to the atmosphere.
5. Cooled and screened sample is unloaded carefully from the sample space 17 through sample outlet 14 to a grinding device to perform grinding of the sample. The side wall 21 of the preparation compartment 12 at the position of the sample outlet 14 is shaped so, that in certain angle it keeps the sample in the sample space 17 while the sample is moved away from the position where the sampling was carried out. However, in this phase when the sampling tool 10 is tilted in slanted or vertical position the sample particles drop out from the sample space 17.
6. After grinding phase the sampling tool 12 is rotated by the tool moving device (i.e., e.g., by twisting the robot arm) such that open end of the analysis compartment 20 is directed upwards. In that position it can be used to collect the sample from the exit of the grinding device. Correct position is shown in the FIG. 2. Sample is then flattened by means a fixed scraper plate by moving the sampling tool in respect of the fixed scraper plate by the tool moving device (i.e., e.g., the robot arm) so that scarper readjusts the sample particles in the analysis compartment 20.
7. The final phase is to determine the amount of residual carbonate from the reburned lime by analyzing the prepared sample. In this phase the sample is taken to contact with analysis probe when the sample lies in the analysis compartment 20. The analysis may be carried out by e.g., using probe that determines the amount of residual carbonate by using Near Infrared Spectroscopy (NIRS). Alternatively, other types of probes being suitable for determining amount of residual carbonate may be applied. Furthermore, the analysis can be carried out by some other suitable methods. For instance, the traditional analysis method i.e., generally used calcinometer method to measure residual carbonate content (e.g., SCAN-32:98), or carbon content-based analysis method to measure residual carbonate content form reburned lime may be alternatively applied.

After the analysis phase the sampling cycle is started over. The phase 1 starts, and the used sample will be dropped into the drop chute when the sampling tool is twisted back into the position wherein it can receive the next lime sample from the chute into the preparation compartment 12. This way all the sample is taken back into the process and any waste is not formed. In case of sampling tool having sieving arrangement which is such that it is capable to screen from the samples only the particles having size over a predetermined upper particle size limit Pₘₐₓ or particles having size under a predetermined lower particle size limit Pₘᵢₙ, sampling and determination of the amount of residual carbonate in a reburned lime sample is accomplished in corresponding manner as described above except that screening phase 2 or 3 is omitted respectively.

The sampling tool as well as the apparatus and the method for determining the amount of residual carbonate in reburned lime sample can be realized further in a way being different from the embodiments described above.

For instance, in some embodiments of the sampling tool the sieving arrangement may comprise a single sieve with adjustable sieve openings. Such sieve may be placed into the preparation compartment, for instance similarly as the first sieve 18 in the embodiment of FIGS. 1-4. Thus, by means of such sieving arrangement it is possible to screen from the sample particles having size over a predetermined upper particle size limit Pmax and under the predetermined lower particle size limit Pₘᵢₙ by carrying out the screening in two separate phases. This would happen such a way that after the particles having size over a predetermined upper particle size limit Pmax has been screened, in the first phase, the size of the sieve openings will be reduced to correspond particle size of the predetermined lower particle size limit Pₘᵢₙ, and then, in the second phase, the preparation compartment will be turned upside down to carry out the said second screening phase by using the sieve with the reduced sieve openings. Turning the preparation compartment upside down also unloads the reject of the first sieving phase (i.e., those too large particles) from above the sieve.

Thus, the invention is restricted to the embodiments described above but it can vary within the scope of the appended claims.

1. A sampling tool for sampling reburned lime from a lime kiln, the sampling tool includes:
a holding device configured to hold a portion of the sampling tool in the lime kiln during the sampling, and
a preparation compartment including:
a sample inlet configured to receive a sample from the lime kiln into the preparation compartment,
a sample outlet configured to unload the sample out from the preparation compartment,
a sieving arrangement configured to confine particles in the sample to a particle size range,
a sample space theconfigured to receive a portion of the sample being screened by the sieving arrangement;
wherein the sieving arrangement comprises a first sieve configured to screen from the sample the particles having size larger than ever an upper particle size limit (Pmax), and a second sieve configured to screen from the sample particles having a particle size smaller than a lower particle size limit (Pmin),
wherein the sample inlet is above the first sieve, and
wherein the sample space is a space between the first sieve and the second sieve.

2. The sampling tool according to claim 1, wherein the preparation compartment comprises a side wall including an upper edge and a lower edge,
wherein the first sieve comprises a first sieve plate and the second sieve comprise a second sieve plate, and
wherein the first sieve plate and the second sieve plate are each attached within the side wall and otherthe first sieve plate is separated from the second sieve plate by a vertical distance.

3. The sampling tool according to claim 2, wherein the first sieve plate of the first sieve is between the upper edge and the lower edge of the side wall,
wherein the second sieve plate of the second sieve is between the sieve plate of the first sieve and the lower edge of the side wall.

4. The sampling tool according to claim 1, wherein the sampling tool further comprises a cooling arrangement configured to reduce the temperature of the sample in the preparation compartment.

5. The sampling tool according to claim 4, wherein the cooling arrangement comprises a cooling medium circulation channel arranged within the preparation compartment.

6. The sampling tool according to claim 1, wherein the sampling tool further comprises an analysis compartment configured to receive the sample from the sample space.

7. The sampling tool according to claim 6, wherein the analysis compartment includes a tray attached to the sampling tool in a position wherein an open end of the tray is perpendicular to or at a slated angle relative to a direction into which the sample flows into the preparation compartment from the lime kiln,
wherein when the sampling tool is in said position to receive the sample from the lime kiln into the preparation compartment, the analysis compartment is in position to unload a previous sample.

8. The sampling tool according to claim 7, wherein the holding device comprises connecting conduit connected to the cooling medium circulation channel and a connector connecting the connecting conduit to a cooling medium supply device.

9. An apparatus for determining amount of residual carbonate in reburned lime sampled from a lime kiln comprising
the sampling tool according to claim 1 and configured to receive a sample of the reburned lime from the lime kiln, and configured to prepare the sample by at least screening the sample,
an analysis compartment,
a sampling tool moving device configured to move the sampling tool into the lime kiln, and from the lime kiln to a grinding device and configured to move the sampling tool with the analysis compartment or the separate analysis device from the grinding device to the residual carbonate analysis device,
the grinding device configured to grind the sample received from the preparation compartment of the sampling tool, and
a residual carbonate analysis device configured to determine an amount of residual carbonate in the sample in the analysis compartment.

10. The apparatus according to claim 9, wherein the sampling tool moving device includes an articulated robot.

11. The apparatus according to claim 9, wherein residual carbonate analysis device is a device configured to determine an amount of residual carbonate based on near infrared spectroscopy.

12. A method for determining an amount of residual carbonate in reburned lime sampled from a lime kiln including:
sampling a reburned lime from a lime kiln by using a sampling tool, wherein the sampling tool includes a holding shaft and a preparation compartment mounted to a distal end of the holding shaft and the preparation compartment includes a sidewall, an upper opening, a first sieve forming a first floor below the upper opening, a second sieve forming a second flow below the first floor, and a sample space between the first floor and the second floor;
preparing a sample of the reburned lime passing through the upper opening and into the preparation compartment, wherein the preparation includes screening the sample to remove particles larger than a first large particle size by passing the sample through the first sieve and into the sample space and screening the sample in the sample space by passing small particles in the sample through the second sieve, wherein screen apertures in the first sieve are larger than screening apertures in the second sieve;
grinding the sample in or from the sample space;
after the grinding, transferring the sample to an analysis compartment and, and in the analysis compartment, determining an amount of residual carbonate in the sample.

13. The method according to claim 12, wherein the determination of the amount of residual carbonate is carried out by applying to the sample in the analysis compartment near infrared spectroscopy, by a calcinometer method to measure the residual carbonate or by a carbon content-based analysis method to measure the residual carbonate.

14. A sampling tool configured to sample reburned lime in a lime kiln, the sampling tool includes:
a holding shaft having a proximal end portion configured to attached to a holding device and a distal end portion configured to be inserted into the lime kiln; and
preparation compartment attached to the distal end of the holding shaft, wherein the preparation compartment includes a sidewall and an upper opening, a first sieve forming a first floor below the upper opening, a second sieve forming a second flow below the first floor, and a sample space between the first floor and the second floor;
wherein reburned lime is received through the upper opening into the preparation component;
wherein the first sieve includes apertures sized to screen and pass down into the sample space particles of the reburned lime having a particle size below an upper particle size limit (Pmax), and a second sieve configured to screen and remove from the sample space particles in the reburned lime having particle size smaller than a lower particle size limit (Pmin).

15. The sampling tool of claim 14, further comprising a cooling medium channel between the second sieve and a bottom of the preparation compartment.

16. The sampling tool of claim 14, further comprising an analysis compartment attached to the sidewall and configured to receive a sample of the reburned lime from the sample space.

17. The sampling tool of claim 14, where a first cross-section of the first sieve in a plane perpendicular to an axis of the holding shaft taken has a curvature such that an apogee of the curvature is vertically lower than the holding shaft and edges of the curvature near the sidewall are higher than the holding shaft.

18. The sampling tool of claim 17, wherein a second cross-section of the second sieve in the plane perpendicular to the axis of the holding shaft taken has a curvature such that an entirety of the curvature is vertically lower than the holding shaft.
